(19) United States
(12) Patent Application Publication
(10) Pub. No.: US 2026/0306389 A1
(43) Pub. Date: Oct. 8, 2026

(54) METHOD OF USING COLOR CHANGING COMPOUNDS IN LIGHT BASED TREATMENTS

(71) Applicant: The Gillette Company LLC, Boston, MA (US)

(72) Inventors: ILARIA AMBROGIO, London (GB); FAIZ FEISAL SHERMAN, Mason, OH (US); SHUFEN FAN, Singapore (SG); RAPHAEL LOUIS MANGIN, Oberursel (DE)

(21) Appl. No.: 19/636,050

(22) Filed: Apr. 1, 2026

Related U.S. Application Data
(60) Provisional application No. 63/783,788, filed on Apr. 4, 2025.

(51) Int. Cl. A61K 8/69; A61Q 9/00; A61Q 19/08

(52) U.S. Cl. CPC A61K 8/69; A61Q 9/00; A61Q 19/08; A61K 2800/26; A61K 2800/438; A61K 2800/45; A61K 2800/592; A61K 2800/81

(57) ABSTRACT

The invention relates to a method for using a composition in conjunction with a light-based device for performing aesthetic treatments such as acne treatment, wrinkle removal, hair removal and other applications based on light treatment. The composition comprises a color changing compound that reacts due to the output of the light-based device.

### FIELD

The invention relates to a method for using a composition in conjunction with a light based device for performing aesthetic treatments such as acne treatment, wrinkle removal, hair removal and other applications based on light treatment.

### BACKGROUND

Known state of art existing methods is characterized by usage of a relatively large light source with a specific wavelength range, which floods a large area, said light source is capable of selective treatment by photothermolysis. For example, hair removal is based on the principle of selectively heating and destroying the hair follicle while avoiding significant damage to surrounding skin or tissue. Hair follicles are selected by photothermolysis an effect based on the fact that hair absorbs great amounts of infrared light, due to its darker color when compared with surrounding skin tissue similar mechanisms will work for other treatment such as stains and veins. Hair is thus automatically selected by the light since it has a darker color and thus higher absorption coefficient. Current technology is based on IPL lamp which emits high energy light pulses to perform the thermolysis treatment. Hair removal is performed by the IR range of light spectrum.

In known hair removal systems treatment is performed by flooding with light a relatively large area with a high intensity pulsed light. Atypical system of a device for dermatological treatment is intended to perform dermatological treatment by intense pulses of light radiated over large skin areas. The treatment selection is performed by chromatic characteristics the skin or hair follicles and selection between treated and not to be treated areas is performed by the light source wavelength selection in a process called photo thermolysis or wavelength depended light absorption.

Light is absorbed by dark objects, so light energy primarily in the IR spectral region can be absorbed by dark material in the skin, but with higher speed and intensity. This dark target matter, or chromophore, can be naturally occurring or artificially introduced. The main drawback of this procedure is that large area of skin is unnecessarily exposed to high levels of intense pulsed light with potential adverse results.

For example, conventional laser hair removal systems rely on flooding large areas with high intensity light, and removal is performed at a wavelength that will not damage the human tissue, where the light intensity will be transmitted by the skin to the follicle depth and destroy the follicle by chromo-thermolysis.

During this process, the only indication for the user to know what portion of skin is treated is their memory, how their skin feels, and testing the perceived completed area by wiping to determine if any hair remains. This can lead to error including missed areas and unnecessary replication of completed areas. As such there is a need to determine, in use, what portions of the skin have been treated to reduce error and repetition.

### SUMMARY

The method described herein utilizes the existing EMR found in light based treatment devices in conjunction with a composition enabled to react when exposed to the EMR. A user applies the composition on the desired treatment area. The composition comprises a color changing compound. The color changing compound may be a photochromic compound which, upon exposure to EMR, changes in color, from either a color to a different color, from a color to colorless, or from colorless to a color, thereby giving the user an indicator of IR exposure to a treated area. After a perceivable change in color, the user may wipe away the composition from the treated area along with any treated hairs that have been removed. The color changing compound may alternatively be a thermochromic compound.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 is flowchart of the representative method.

FIG. 2A-2C are representative photochromic compounds.

FIG. 3A-3G are representative photochromic compounds.

### DETAILED DESCRIPTION

All ingredient percentages described herein are by weight of the composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. Unless otherwise indicated, all measurements are understood to be made at approximately 25° C. and at ambient conditions, where “ambient conditions” means conditions under about 1 atmosphere of pressure and at about 50% relative humidity. All numeric ranges are inclusive of narrower ranges not explicitly delineated within these ranges, and delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods. As used in the description and the appended claims, the singular forms “a,” “an,” and “the” are intended to include the plural forms as well, unless the context clearly indicates otherwise.

### Definitions

“About” modifies a particular value by referring to a range of plus or minus 20% or less of the stated value (e.g., plus or minus 15% or less, 10% or less, or even 5% or less).

“Activate” and variations thereof, when referring to a photochromic dye or a composition comprising a photochromic dye, means causing the photochromic dye and/or composition to change color by exposing it to electromagnetic radiation (“EMR”) of a particular wavelength or changing the temperature of the dye.

“Color” refers to visibly perceived light of a particular wavelength, generally between 400 nm and 700 nm. Color may be characterized in terms of Hue, Saturation, and Lightness, and can be measured using conventional methods, for example, with a spectrophotometer or colorimeter.

“Cosmetic agent” means any substance, as well any component thereof, intended to be rubbed, poured, sprinkled, sprayed, introduced into, or otherwise applied to a mammalian body or any part thereof to provide a cosmetic effect. Cosmetic agents may include substances that are Generally Recognized as Safe (GRAS) by the U.S. Food and Drug Administration.

“Deactivate” and variations thereof, when referring to a photochromic dye or a composition comprising a photochromic dye, means causing the photochromic dye and/or composition to revert back to substantially the same color it was prior to activation by exposing the dye to EMR of a particular wavelength, removing activating EMR or changing the temperature of the dye.

“Different colors” and variations thereof mean colors that have a different Hue, Saturation and/or Lightness value relative to one another. In some aspects, different colors can be characterized by having a ΔE of greater than 1, relative to one another, according to the Color Measurement method.

“Fluence” means the time-integrated flux of EMR (i.e., activating EMR and deactivating EMR) on an irradiated surface. Fluence can be readily determined by those skilled in the art.

“Light sensitive dye” is a dye that has a chemical reaction when exposed to light.

“Suitable for application to human hair” means that the composition or components thereof, are acceptable for use in contact with human hair and the scalp and skin without undue toxicity, incompatibility, instability, and the like.

“Substantially free of” means a composition or ingredient contains less than 3% (e.g., less than 2%, 1% or even less than 0.5%) of a subject material, by weight of the composition or ingredient. “Free of” means 0% of a subject material is present in the composition or ingredient.

“Tunable” means that the hair color change provided by the photochromic hair dye compositions herein can be adjusted to a desired color. The hair color change may be tuned, for example, by changing: the activating/deactivating time, the strength of the EMR source, the concentration or ratio of photochromic dyes and/or the activating/deactivating technique (e.g., how close the EMR source is to the hair or selectively exposing target portions of hair to the EMR source).

“Visible color change” and variations thereof mean a color change that results in a ΔE of more than 1 (e.g., 2, 3, 5, 10, 20 or more). ΔE can be determined according to the Color Measurement method described in more detail below.

Reference will now be made in detail to the present embodiments of the present invention, examples of which are illustrated in the accompanying drawings, wherein like reference numerals refer to the like elements throughout. The embodiments are described below in order to explain the present invention by referring to the figures.

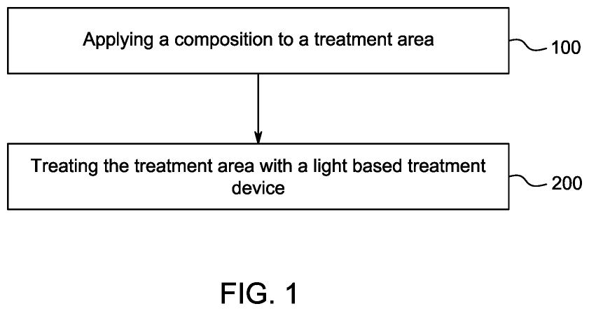

FIG. 1 shows a flowchart representing each step of the method. The method comprising: Applying a composition to a treatment area 100 and treating the treatment area with a light based treatment device 200. The composition comprises a color changing compound. The step of applying a composition may be done using a hand, a substrate, or any tool that allows for delivery of the composition onto the skin. The step of applying a composition may further comprise massaging, either by hand or by utilizing a tool, the composition into the skin. The step of treating the treatment area with a light based treatment device may be done according to the instructions provided by the manufacturer of the light based treatment device utilized.

The method may further include wiping the treatment area. Wiping the treatment area may be done by a hand, absorbent material, substrate, or tool allowing for the removal of composition. The composition comprises one or more of a photochromic dye. The step of treating the treatment area with a light based treatment device 200 may further comprise treating the treatment area until the composition exhibits a Delta E of at least 0.5, such as, for example, 0.5, 1, 1.5, 2, 2.5, 3, 3.5, 4, 4.5, 5, 5.5, 6, 6.5, 7, 7.5, 8, 8.5, 9, 9.5, or 10. As used herein, Delta E is the difference between two colors designated as two points in the Lab color space. With values assigned to each of the L, a, and b attributes of two colors, utilizing geometry to calculate the distance between their two placements in the Lab color space.

The color changing compound may be any one of one or morephotochromic compounds, one or more thermocromic compounds or any combination thereof.

### Photochromic Compound

Photochromic compounds (or dyes) are generally recognized as compounds that undergo a reversible color change when exposed to EMR of a particular wavelength and fluence (“activating EMR”). Photochromic compounds are classified into two types, p-type and t-type. When a photochromic compound is exposed to an activating and/or deactivating EMR it undergoes a molecular transformation between isomers of the photochromic compound molecule, for example between a base (unactivated) state for the compound and an activated analog of the compound in a different color state as described in more detail below. In some case, prolonged exposure to activating EMR can further transform the photochromic compound to a dyotropic rearrangement of the compound (e.g., from the activated analog), which is a generally permanent, stable, and/or irreversible transformation of the compound to a third color state (e.g., same or different from that of the activated analog), as described in more detail below.

P-type photochromic compounds are generally thermally irreversible, but photochemically reversible. In other words, a p-type photochromic compound will change color when irradiated with activating EMR but will generally not revert back to its original color when the activating EMR is removed or when the temperature of the dye changes. The p-type photochromic compound suitable for use herein can be activated by an activating EMR having a wavelength of 250 nm to 395 nm (e.g., 260 nm to 380 nm or 270 nm to 370 nm) and a fluence of 1 mJ/cm² to 50 J/cm². A p-type photochromic dye is deactivated by exposing the dye to deactivating EMR, which has a different wavelength from the activating EMR. In some embodiments, the deactivating EMR has a wavelength of 400 to 1400 nm (e.g., 400 to 700 nm; 425 nm to 650 nm) and a fluence of 1 mJ/cm² to 50 J/cm². The photochromic compounds suitable for use herein may have an activation and/or deactivation time of less than 1 second up to 10 minutes (e.g., 0.001 seconds to 5 minutes, 0.01 second to 3 minutes, 0.1 second to 2 minutes seconds, 0.5 second to 90 seconds, or even 10 seconds to 60 seconds). Relatively shorter activation and/or deactivation times may be more convenient to a user and therefore preferred.

Photochromic compounds according to the disclosure include p-type fluorine-free photochromic compounds that can exist in one, two, or three related states or forms: a fluorine-free first compound, a fluorine-free second compound that is an activated analog of the fluorine-free first compound, and a fluorine-free third compound that is a dyotropic rearrangement of the fluorine-free first compound (or of the fluorine-free second compound). The fluorine-free first compound has a cyclic bridge (e.g., having 4, 5, or 6 ring atoms) with one ethylenically unsaturated bond (C═C). The fluorine-free first compound further includes first and second substituents (same or different) bonded to opposing carbon atoms in the ethylenically unsaturated bond. The first substituent includes a first unsaturated heterocycle (e.g., thiophene or otherwise) having a first aromatic group or a first halogen (e.g., Cl, F) bonded to the first unsaturated heterocycle, for example at a carbon atom of the heterocycle. The second substituent includes a second unsaturated heterocycle (e.g., thiophene or otherwise) having a second aromatic group bonded to the second unsaturated heterocycle, for example at a carbon atom of the heterocycle. The first and second aromatic groups can be substituted with a linear or branched alkyl or alkylene oxide group, a hydroxyl group, an amino or ammonium group, and combinations thereof, for example including one or more of the various linking groups described below between the aromatic ring and the aromatic substituent(s). The fluorine-free first compound can include the Formula (I) compound described below. The activated analog structure and the dyotropic rearrangement structure for the fluorine-free second and third compounds, respectively, are illustrated below for the representative case of the Formula (I) base/unactivated structure. The first and second fluorine-free compounds are thermally irreversible, but photochemically reversible between each other with application of activating or deactivating EMR. The transformation of the second fluorine-free compound to the third fluorine-free compound is thermally and photochemically irreversible.

A photochromic compound according to the disclosure can be represented by Formula (I), which represents the unactivated or base state of the photochromic compound. As described above, the photochromic compound can exist in multiple related states: the unactivated or base state of Formula (I), an activated analog of Formula (I) (e.g., reversibly activated by UV EMR), and a dyotropic rearrangement Formula (I) or its activated analog (e.g., irreversibly activated by further UV EMR). Formula (I) is illustrated below as having a bridge B and two substituents A¹ and A² at the ethylenic bond of the bridge, where A¹ and A² can have the aromatic-substituted heterocycle or the halogen-substituted heterocycle structures of Formulas (Ia) and (Ib), respectively:

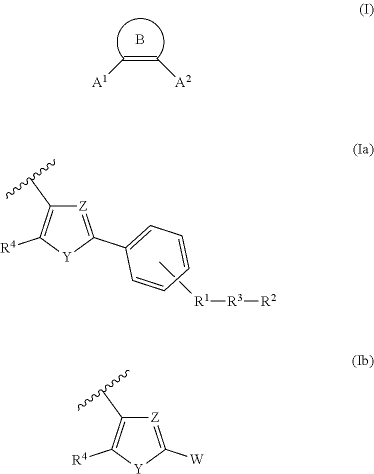

In Formula (I), the bridge or ring B has the formula

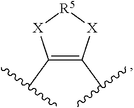

X is CH₂ and R⁵ is a bond (i.e., absent) or unsubstituted or substituted C₁₋₂ alkylene (e.g., 1 or 2 carbon atoms); or X is carbonyl and R⁵ is O, NH, or NR⁸. R⁸ is independently selected from H, C₁₋₆ alkyl (e.g., 1, 2, 3, 4, 5, or 6 carbon atoms), and aryl. The bridge can have 4, 5, or 6 ring atoms. Illustrative structures for the ring B are

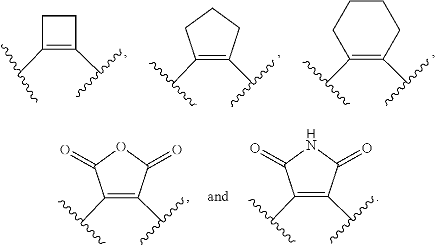

In Formula (I), A¹ and A² can independently be represented by Formula (Ia) and or Formula (Ib), with the proviso that at least one of A¹ and A² is a substituent having Formula (Ia). In some embodiments, A¹ is identical to A², and both are represented by Formula (Ia).

In Formulas (Ia) and (Ib), each Y is independently selected from S, O, and NR⁶; and each Z is independently selected from N and CR⁷. R⁶ and R⁷ are independently selected from H, C₁₋₆ alkyl (e.g., 1, 2, 3, 4, 5, or 6 carbon atoms), and aryl. In embodiments, Y is S and Z is CH to represent a thiophene heterocycle. Each R⁴ is independently selected from C₁₋₈alkyl (e.g., 1, 2, 3, 4, 5, 6, 7, or 8 carbon atoms), C₄₋₈cycloalkyl (e.g., 4, 5, 6, 7, or 8 carbon atoms), C₁₋₁₂alkoxy (e.g., 1, 2, 3, 4, 5, 6, 7, 8, 9, 10, 11, or 12 carbon atoms), C≡CR⁷, and cyano.

In Formula (Ia), each R¹ is independently selected from O, S, C═C, C≡C, OC(═O), C(═O)O, C(═O)S, SC(═O), C(═O)NH, NHC(═O), C(═S)NH, NHC(═S), NHC(═O)NH, and NHC(═S)NH. For each of the foregoing groups, the left side of the group can be that which is bonded to the aromatic ring, and the right side of the group can be that which is bonded to R³—R². For example, when R¹ is C(═O)O, then the corresponding structure in Formula (Ia) can be represented by [aromatic ring]—C(═O)O—R³—R². Each R² is independently selected from H, C₁₋₆alkyl (e.g., 1, 2, 3, 4, 5, or 6 carbon atoms), N(R⁸)₂, N⁺H(R⁸)₂, and any of the foregoing linked to R³ via an additional R¹ group. R⁸ is independently selected from H, C₁₋₆ alkyl (e.g., 1, 2, 3, 4, 5, or 6 carbon atoms), and aryl. Each R³ is independently a bond (i.e., absent such that R¹ and R² are directly bonded), linear or branched C₁₋₂₀alkylene (e.g., 1, 2, 3, 4, 5, 6, 7, 8, 9, 10, 11, 12, 13, 14, 15, 16, 17, 18, 19, or 20 carbon atoms), or linear or branched (OCH₂CH₂)ₙ, where n is an integer in the range of 1 to 20 (e.g., 1, 2, 3, 4, 5, 6, 7, 8, 9, 10, 11, 12, 13, 14, 15, 16, 17, 18, 19, or 20 ethylene oxide units).

Each m is independently 1 to 5, for example 1, 2, 3, 4, or 5 of the same or different —R¹—R³—R² substituents attached to the aromatic or phenyl (Ph) group at any combination of ortho, meta, and para locations relative to the bond to the heterocycle.

Suitably, the —R¹—R³—R² substituent is bonded to the aryl ring at the para position relative to the bond to the 5-member heterocycle. In other embodiments, the R¹—R³—R² substituent can be bonded at ortho or meta positions relative to the bond to the 5-member heterocycle.

Formula (Ia) is illustrated with a single —R¹—R³—R² substituent. More generally, the aromatic ring in Formula (Ia) can be substituted with up to five substituents (same or different), for example being represented by [R¹—R³—R²]n, where n=1, 2, 3, 4, or 5 and R¹, R³, and R² can be independently selected from the options above for each n.

In Formula (Ib), W is as halogen, suitably Cl, Br, or I.

The photochromic compound can include any combination or distribution of its different forms, including its unactivated base form (e.g., Formula (I) compound), activated analog form (e.g., activated analog of Formula (I)), and dyotropic rearrangement form (e.g., dyotropic rearrangement of Formula (I)). Each form can independently be absent, substantially absent, or present in any amount relative to all forms combined, for example with each form independently being present in an amount of at least and/or up to 0, 1, 2, 5, 10, 20, 25, 30, 40, 50, 60, 70, 75, 80, 90, 95, 98, 99, or 100 mol. % and ranges therebetween relative to all three forms. Alternatively, or additionally, any two forms can be present in relative molar ratios of 1:99 to 99:1, or 5:95 to 95:5, or 10:90 to 90:10, or 25:75 to 75:25.

Photochromic compounds and corresponding coloring compositions of the disclosure can be activated by exposure to an activating EMR. Activating a photochromic compound of the disclosure can convert some, substantially all, or all of the compound to an activated state (e.g., second or third fluorine-free compound). The activating EMR can include EMR of one or more wavelengths that are absorbed by the compound. The activating EMR can include EMR having wavelengths in a range of 100 nm to 400 nm, or 250 nm to 395 nm, or 250 nm to 350 nm, or 260 nm to 340 nm, or 270 nm to 330 nm, or 280 nm to 320 nm. The wavelength of the activating EMR can be about 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 395 nm, for example, or within a range formed by any such values as endpoints. The activating EMR can also include EMR having wavelengths that are not absorbed by the compound. The source of the activating EMR is not particularly limited, for example including a lamp, LED, laser, etc. emitting light at a specific wavelength or range of wavelengths.

Similarly, activated analogs of photochromic compounds of the disclosure (e.g., second fluorine-free compound) can be deactivated by exposure to a deactivating EMR. Deactivating the activated analog of a compound of the disclosure can convert some, substantially all, or all of the activated analog to the compound's structure prior to activation (e.g., first fluorine-free compound). The deactivating EMR can include EMR of one or more wavelengths that are absorbed by the activated analog of the compound of the disclosure. The deactivating EMR can comprise EMR having wavelengths in a range of 400 nm to 1400 nm, or 400 nm to 1200 nm, or 400 nm to 1000 nm, or 400 nm to 900 nm, or 400 nm to 800 nm, or 400 nm, to 700 nm, or 450 nm to 650 nm, or 500 nm to 600 nm. The wavelength of the deactivating EMR can be about 400 nm, 425 nm, 450 nm, 475 nm, 500 nm, 525 nm, 550 nm, 575 nm, 600 nm, 625 nm, 650 nm, 675 nm, 700 nm, 800 nm, 900 nm, 1000 nm, 1100 nm, 1200 nm, 1300 nm, or 1400 nm for example, or within a range formed by any such values as endpoints. The deactivating EMR can include EMR having one or more wavelengths that are absorbed by the activated analog. The deactivating EMR can also include EMR having wavelengths that are not absorbed by the activated analog. The source of the deactivating EMIR is not particularly limited, for example including a lamp, LED, laser, etc. emitting light at a specific wavelength or range of wavelengths. For instance, the source of the deactivating EMR can be a white light source.

Compounds of the disclosure may exhibit a 50% activation time, denoted herein as t_{1/2,a}. A 50% activation time of a sample including a compound of the disclosure is the time in which the absorbance of the sample increases to 50% of the final or asymptotic absorbance of the sample after prolonged exposure to activating EMR, for example after conversion of substantially all of the compound to its activated analog upon exposure to activating EMR, or after conversion of a sufficient portion of the compound to its activated analog to achieve it final or most intense color state. The 50% activation time is a measure of the rate of formation of the activated analog and is accordingly a measure of the rate of color change the compound undergoes upon exposure to activating EMR. Absorbance for determining a 50% activation time can be measured at any wavelength at which the activated analog absorbs, such as the wavelength of peak absorbance of the activated analog. The 50% activation time is preferably on the order of seconds. The 50% activation time can be 30 seconds or less, or in a range of 1 to 600 seconds, for example at least and/or up to 1, 2, 3, 5, 7, 10, 15, 20, 30, 40, 50, 60, 90, 120, 180, 240, 300, 360, 480, or 600 seconds and ranges therebetween.

Compounds of the disclosure may exhibit a 50% deactivation time, denoted herein as t_{1/2,d}. A 50% deactivation time of a sample including the activated analog of a compound of the disclosure is the time in which the absorbance of an initially fully activated sample decreases to 50% of its initial absorbance upon exposure to deactivating EMR. The 50% deactivation time is a measure of the rate of disappearance of the activated analog and is accordingly a measure of the rate of color change the activated analog undergoes upon exposure to deactivating EMR. Absorbance for determining a 50% deactivation time can be measured at any wavelength at which the activated analog absorbs, such as the wavelength of peak absorbance of the activated analog. The 50% deactivation time is preferably on the order of minutes. The 50% deactivation time can be 30 minutes or more, or in a range of 2 to 600 minutes, for example at least and/or up to 2, 3, 5, 7, 10, 15, 20, 30, 40, 50, 60, 90, 120, 180, 240, 300, 360, 480, or 600 minutes and ranges therebetween.

The photochromic compound of the disclosure can be non-phototoxic at concentrations up to 100 μg/mL as measured by the OECD 432 Assay (NRU uptake phototoxicity test). Alternatively, or additionally, the photochromic compound of the disclosure can be non-photoreactive at concentrations up to 100 μg/mL as measured by the OECD 495 Assay (ROS photoreactivity test). Other concentration thresholds can be used for either test, for example 1, 2, 5, 10, 20, 50, 100, 200, 500, or 1000 μg/mL.

The composition may comprise two different photochromic dyes, a cosmetically acceptable solvent and a carrier, as well as other optional ingredients commonly found in lotions as described below and the like. The photochromic dyes may each be present in the composition at 0.001% to 25% (e.g., 0.05% to 20%, 0.1% to 10%, 0.5% to 5%, or even 1% to 3%). The composition also includes a suitable solvent to ensure the dyes are suitably dispersed in the carrier. The solvent may be present at 1% to 99.99% (e.g., 2% to 90%, 3% to 80%, 4% to 70%, or even 5% to 50%). The pH of the compositions herein may be between 2 and 10 (e.g., 3, 4, 5, 6, 7, 8 or 9).

In some embodiments utilizing more than one photochromic dyes, the photochromic dyes are included in the composition at a ratio of first dye to second dye of 1:50 to 50:1 (e.g., 1:30, 1:20, 1:10, 1:5, 1:3, 1:2, 1:1, 2:1, 3:1, 5:1, 10:1, 20:1, or 30:1). Of course, one or more additional photochromic dyes may also be included at a suitable ratio to the first and/or second dye. It is to be appreciated that the ratio of the photochromic dyes in the composition can be important visible change in color during treatment.

In some instances, the p-type photochromic dyes used herein include those belonging to the diarylethene (“DAE”) family and/or fulgide family. Some non-limiting examples of diarylethene and fulgide photochromic dyes are described in U.S. Pat. No. 6,123,952 and US20120017929. Some particularly suitable examples of P-type photochromic dyes for use herein include DAE-0001 (cyan), DAE-0012 (magenta), DAE-0068 (yellow) from Yamada Chemical Company. The invention may further comprise, or utilize in a similar manner, the photochromic dyes described in “Photo-Chromeleon: Re-Programmable Multi-Color Textures Using Photochromic Dyes” published in UIST' 19, Oct. 20-23, 2019, New Orleans LA, USA.

The photochromatic dyes may comprise of dyes such as those described in “Synthesis and Properties of Photochromic Diarylethenes with Heterocyclic Aryl Groups” by Masahiro Irie and Kingo Uchida published in Bull. Chem. Soc. Jpn., 71, 985-996 (1998).

Other potential photochromic compounds include, for example, those described in U.S. Pat. Nos. 4,205,988 and 7,452,950 which include, without limitation, diaryl ethenes, fulgides, fulgimides, phenoxy derivatives of phenoxy naphthacene quinone and others.

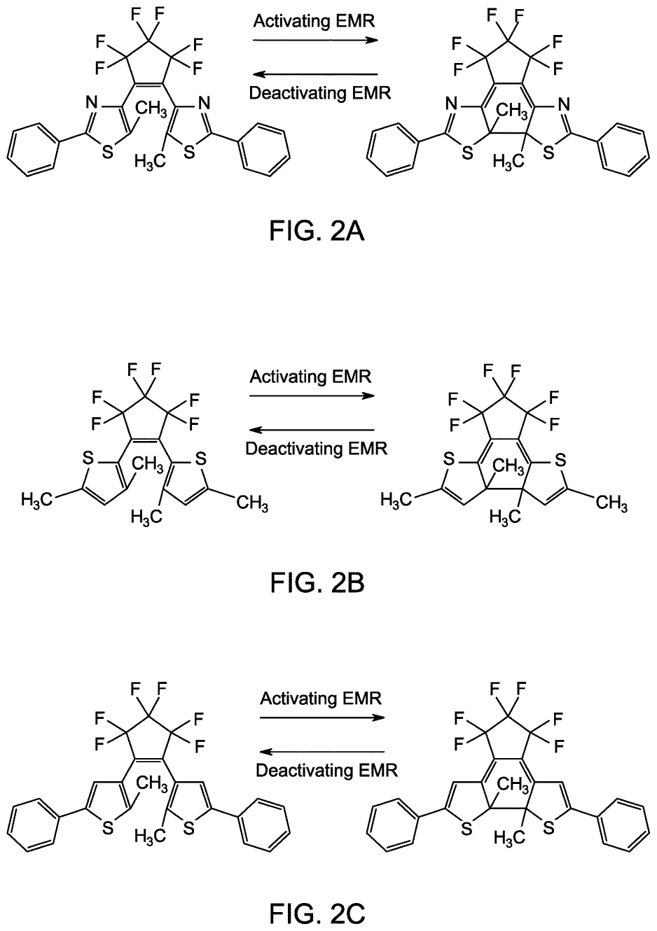

FIGS. 2A, 2B, and 2C depict the chemical structures of diarylethene photochromic dyes. FIG. 2A depicts 4,4′-(perfluorocyclopent-1-ene-1,2-diyl)bis(5-methyl-2-phenylthiazole), which is a P-type photochromic dye that changes from clear to magenta when exposed to a suitable activation energy. FIG. 2B depicts 5,5′-(perfluorocyclopent-1-ene-1,2-diyl)bis(2,4-dimethylthiophene), which is a p-type photochromic dye that changes from clear to yellow when exposed to a suitable activation energy. FIG. 2C depicts 3,3′-(perfluorocyclopent-1-ene-1,2-diyl)bis(2-methyl-5-phenylthiophene), which is a p-type photochromic dye that changes from clear to cyan when exposed to a suitable activating energy. In FIGS. 2A, 2B and 2C, the structures on the left-hand side illustrate the unactivated state of the dye, and the structures on the right-hand side illustrate the activated state of the dye. As illustrated in the figures, the activated dyes have a closed ring structure and the unactivated dye have an open ring structure. The wavelength of the activating EMR for the photochromic dyes illustrated in FIGS. 2A, 2B, and 2C can range from 250 nm to 395 nm, and the wavelength of the deactivating EMR can range from 400 nm to 700 nm, depending on the dye. The fluence of the activating and deactivating EMR of the photochromic dyes can be 1 mJ/cm² to 50 J/cm².

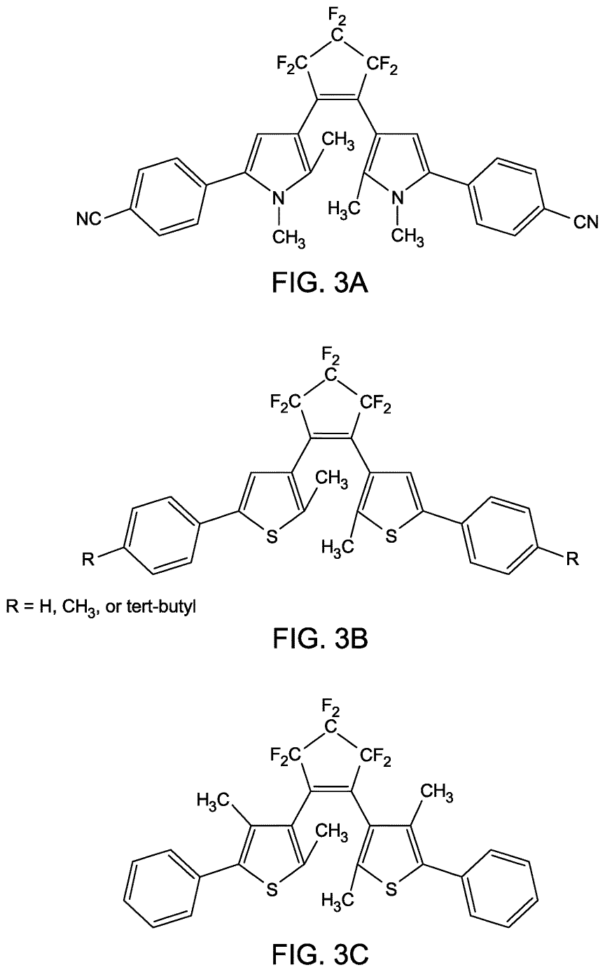

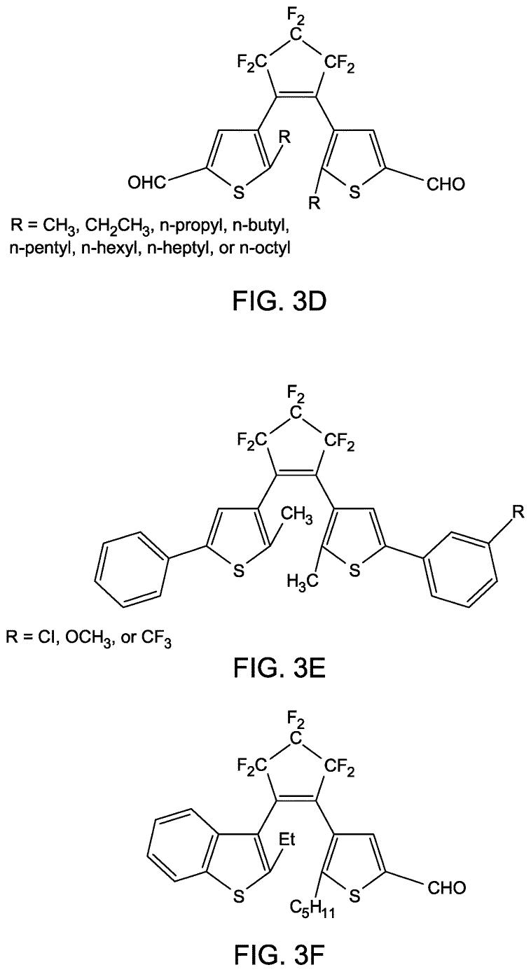

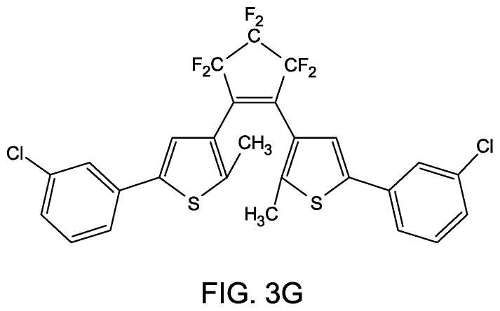

FIGS. 3A to 3G depict additional examples of blue photochromic dyes (i.e., dyes that generally appear blue to the human eye when activated by an activating EMR).

In some embodiments, the hair dye compositions herein may include one or more t-type photochromic dyes. T-type photochromic dyes are thermally reversible, which generally means they will revert back to their original color once the activating EMR is removed. T-type photochromic dyes include azobenzene and spiropyran compounds. Some non-limiting examples of T-type photochromic dyes can be found in U.S. Pat. Nos. 5,581,090, 5,730,961, and FR1604929.

### Thermochromic Compounds

The composition may comprise one or more thermochromic compounds such as, for example and without limitation, leuco dyes, inorganic materials, or combinations thereof, such as, for example and without limitation, titanium dioxide, zinc sulfide, zinc sulfide, and zinc oxide. The thermochromic compounds may be a combination of any of the materials or compounds listed above.

The thermochromic compound may each be present in the composition at 0.001% to 25% (e.g., 0.05% to 20%, 0.1% to 10%, 0.5% to 5%, or even 1% to 3%).

The composition may further comprise any of a fatty substance, emulsifying agents, thickening agents, additives, or combinations thereof. The composition may be in the form of a cosmetically acceptable medium such as a carrier as described below.

The composition herein can include 1%-95% of a cosmetically acceptable carrier. The carrier will typically be liquid, but embodiments in which the carrier is solid or semi-solid (e.g., gel) or other cosmetically acceptable forms are also contemplated herein. The carrier may include polar and/or non-polar materials and may be in the form of a single phase (e.g., solution or dispersion) or multiple phases (e.g., an oil-in-water or water-in-oil emulsion). In some embodiments, the carrier includes a solvent in which the photochromic compounds are soluble. The solvent may make up some or all of the carrier (e.g., 5% to 100%). In some embodiments, the photochromic compound has a solubility of 1 mg/L to 500 g/L in the solvent, for example at least and/or up to 0.001, 0.01, 0.1, 1, 2, 5, 10, 20, 50, 100, 200, 300, 400, or 500 g/L and ranges therebetween in the solvent at about 20° C., 25° C., 40° C., or 50° C. Some non-limiting examples of solvents that may be suitable for dissolving DAE photochromic dyes include emulsifiers (e.g., TWEEN® 20, TWEEN® 40, TWEEN® 60, and TWEEN® 80 brand polysorbate and SPAN® 20, SPAN® 40, SPAN® 60 and SPAN® 80 brand sorbitan laurate), fatty acids (e.g., steric acid, oleic acid, and palmitic acid), fatty alcohols (e.g., cetyl alcohol, lauryl alcohol, stearyl alcohol, oleyl alcohol, and octyldodecanol), fatty esters (e.g., caprylic/capric triglyceride, isopropyl palmitate, and isopropyl myristate), silicones (e.g., cyclopentasiloxane, cyclohexasiloxane, dimethicone, and methicone) and botanical oils (e.g., wheatgerm oil, beauty-leaf oil, sesame oil, macadamia oil, grapeseed oil, rapeseed oil, coconut oil, groundnut oil, palm oil, castor oil, jojoba oil, olive oil or cereal germ oil, cedarleaf oil, turpentine oil, and eucalyptus oil) and combinations of these. Botanical oils may be particularly suitable because they can be naturally and/or sustainably sourced and are generally perceived by consumers as being better for their body.

The compositions of the present invention comprise at least one fatty substance chosen from fatty acids and fatty alcohols.

Preferably, the at least one fatty substance chosen from fatty acids and fatty alcohols is a liquid at room temperature (25 degrees C.) and at atmospheric pressure (760 mmHg).

The fatty alcohols may be chosen from linear, branched or cyclic, saturated or unsaturated, fatty alcohols comprising 6 to 50 carbon atoms and comprising one or more OH, optionally comprising one or more NH2.

Mention may in particular be made of: linear or branched, saturated or unsaturated, C6-050, especially C6-C32, in particular C8-C28, monoalcohols, and in particular isostearyl alcohol, cetyl alcohol, cetearyl alcohol, oleyl alcohol, isopalmitoyl alcohol, lauryl alcohol, myristyl alcohol, 2-butyloctanol, 2-hexyldecanol, 2-octyldecanol, 2-octyldodecanol, 2-octyltetradecanol, 2-decyl-tetradecanol, 2-dodecylhexadecanol, and in particular the alcohols sold under the name Jarcol™ by the company Jarchem Industries, such as Jarcol™ 1-12, Jarcol™ 1-16, Jarcol™ I-20 and Jarcol™ I-24;
linear or branched, saturated or unsaturated, C6-050, especially C6-C40, in particular C8-C38, and especially branched C32-C36, diols, and in particular the commercial product Pripol™ 2033 from Uniqema;
linear or branched, saturated or unsaturated, C6-050, especially C6-C32, in particular C8-C28, triols, and in particular phytantriol; Preferred fatty alcohols of the present invention may be chosen from cetyl alcohol, cetearyl alcohol, isostearyl alcohol, and oleyl alcohol.

The fatty acids of the present invention may be chosen from the acids of formula RCOOH, in which R is a saturated or unsaturated, linear or branched radical preferably comprising from 7 to 39 carbon atoms.

Preferably, R is a C7-C29 alkyl or C7-C29 alkenyl group and better still a C12-C24 alkyl or C12-C24 alkenyl group. R may be substituted with one or more hydroxyl groups and/or one or more carboxl groups.

The fatty acids may be chosen from C12-C22 higher fatty acids, such as stearic acid, myristic acid, oleic acid, linoleic acid, linolenic acid, lauric acid, palmitic acid, and mixtures thereof.

In preferred embodiments, the fatty substances of the compositions of the present invention comprise fatty acids.

In other preferred embodiments, the fatty substances of the compositions of the present invention comprise fatty acids and fatty alcohols.

The fatty sub stance(s) are present in the composition in an amount ranging from about 0.1 to about 20% by weight, preferably in an amount ranging from about 0.25 to about 10% by weight, more preferably still in an amount ranging from about 0.25 to about 5% by weight, and even more preferably still in an amount ranging from about 0.5 to about 3% by weight, based on the total weight of the composition, including all ranges and subranges therebetween.

### Emulsifying Agents

The compositions according to the invention comprise at least one emulsifying agent is chosen from oxyethylenated and/or oxypropylenated ethers of glycerol, oxyethylenated and/or oxypropylenated ethers of fatty alcohols, fatty acid esters of polyethylene glycol, fatty acid esters of oxyethylenated and/or oxypropylenated glyceryl ethers, fatty acid esters of polyols and mixtures thereof.

According to the invention, the at least one emulsifying agent is appropriately chosen to obtain a composition that is an emulsion, preferably, an oil-in-water emulsion. In particular, the emulsifying agent has, at 25 degrees centigrade, an HLB (hydrophilic-lipophilic balance), in the Griffin sense, of greater than or equal to 8.

The emulsifying agents of the present invention comprise nonionic surfactants and are further described as follow:
a) nonionic surfactants with an HLB of greater than or equal to 8 at 25 degrees centigrade, used alone or as a mixture; mention may be made especially of:
   oxyethylenated and/or oxypropylenated ethers (which may comprise from 1 to 150 oxyethylene and/or oxypropylene groups) of glycerol;
   oxyethylenated and/or oxypropylenated ethers (which may comprise from 1 to 150 oxyethylene and/or oxypropylene groups) of fatty alcohols (especially of C8-C24 and preferably C12-Ci8 alcohol), such as oxyethylenated cetearyl alcohol ether containing 30 oxyethylene groups (CTFA name Ceteareth-30), oxyethylenated stearyl alcohol ether containing 20 oxyethylene groups (CTFA name Steareth-20) and the oxyethylenated ether of the mixture of C12-C15 fatty alcohols comprising 7 oxyethylene groups (CTFA name C12-15 Pareth-7) sold under the name Neodol 25-7(R) by Shell Chemicals;
   fatty acid esters (especially of a C8-C24 and preferably Ci6-C22 acid) of polyethylene glycol (which may comprise from 1 to 150 ethylene glycol units), such as PEG-100 stearate or PEG-50 stearate and PEG-40 monostearate sold under the name Myrj™ 52P by the company ICI Uniqema, or else PEG-30 glyceryl stearate sold in particular under the name Tagat® S by the company Evonik Goldschmidt, or else PEG-150 distearate sold in particular under the name KESSCO® PEG 6000 DS by the company ITALMATCH CHEMICALS ARESE;
   fatty acid esters (especially of a C8-C24 and preferably Ci6-C22 acid) of oxyethylenated and/or oxypropylenated glyceryl ethers (which may comprise from 1 to 150 oxyethylene and/or oxypropylene groups), for instance PEG-200 glyceryl monostearate sold under the name Simulsol® 220™ by the company SEPPIC; glyceryl stearate polyethoxylated with 30 ethylene oxide groups, for instance the product Tagat® S sold by the company Evonik Goldschmidt, glyceryl oleate polyethoxylated with 30 ethylene oxide groups, for instance the product Tagat® 0 sold by the company Evonik Goldschmidt, glyceryl cocoate polyethoxylated with 30 ethylene oxide groups, for instance the product Varionic® LI 13 sold by the company Sherex, glyceryl isostearate polyethoxylated with 30 ethylene oxide groups, for instance the product Tagat® L sold by the company Evonik Goldschmidt, and glyceryl laurate polyethoxylated with 30 ethylene oxide groups, for instance the product Tagat® I from the company Evonik Goldschmidt;
b) nonionic surfactants with an HLB of less than 8 at degrees centigrade, optionally combined with one or more nonionic surfactants with an HLB of greater than 8 at 25 degrees centigrade, such as those mentioned above; mention may be made especially of:
   oxyethylenated and or oxypropylenated ethers (which may comprise from 1 to 150 oxyethylene and/or oxypropylene groups) of fatty alcohols (especially of C8-C24 and preferably C12-C)g alcohols) such as the oxyethylenated ether of stearyl alcohol containing two oxyethylene groups (CTFA name Steareth-2);
   fatty acid esters (especially of a C8-C24 and preferably Ci6-C22 acid) of polyols, especially of glycerol or of sorbitol, such as glyceryl stearate, glyceryl stearate such as the product sold under the name Tegin® M by the company Evonik Goldschmidt, glyceryl laurate such as the product sold under the name Imwitor® 312 by the company Hiils, polyglyceryl-2 stearate, sorbitan tristearate or glyceryl ricinoleate.

According to one particular embodiment of the invention, the at least one emulsifying agent of the present invention comprises glyceryl esters and polyethylene glycol esters of fatty acids.

According to another particular embodiment of the invention, the at least one emulsifying agent of the present invention is chosen from PEG-150 distearate sold under the name of Kessco PEG® 6000 DS from ITALMATCH CHEMICALS ARESE, Glyceryl stearate (and) PEG-100 Stearate (for example such as the product sold under name ARLACEL™ 165 from CRODA); and mixtures thereof.

The composition according to the invention may contain from about 0.1 to about 10% by weight, or from about 0.25 to about 5% by weight of total emulsifying agent(s), based on the total weight of the said composition, including all ranges and subranges therebetween.

### Thickening Agents

The at least one thickening agent of the present invention are preferably chosen from polymers of natural origin and may include, for example, thickening polymers comprising at least one sugar unit, for instance nonionic guar gums, optionally modified with C1-C6 hydroxyalkyl groups; biopolysaccharide gums of microbial origin, such as scleroglucan gum or xanthan gum; gums derived from plant exudates, such as gum arabic, ghatti gum, karaya gum, gum tragacanth, carrageenan gum, agar gum and carob gum; pectins; alginates; starches; hydroxy(C₁-C6)alkylcelluloses and carboxy(C1-C6)alkylcelluloses.

It should be noted that the term “sugar unit” denotes a monosaccharide (i.e. monosaccharide or oside or simple sugar) portion, an oligosaccharide portion (short chains formed from a sequence of monosaccharide units, which may be different) or a polysaccharide portion [long chains consisting of monosaccharide units, which may be different, i.e. polyholosides or polyosides]. The saccharide units may also be substituted with alkyl, hydroxyalkyl, alkoxy, acyloxy or carboxyl radicals, the alkyl radicals containing from 1 to 4 carbon atoms.

Non-limiting examples of nonionic, unmodified guar gums that may be used in various embodiments include Guargel D/15 (Noveon); Vidogum GH 175 (Unipectine), Meypro-Guar 50 and Jaguar C (Meyhall/Rhodia Chimie). Non-limiting examples of modified nonionic guar gums include Jaguar HP8, HP60, HP120, DC 293 and HP 105 (Meyhall/Rhodia Chimie); Galactasol 4H4FD2 (Aqualon).

Among these gums, mention will be made of scleroglucans such as, for example, Actigum CS from Sanofi Bio Industries; Amigel from Alban Muller International, and also the glyoxal-treated scleroglucans described in FR2633940); xanthan gums, for instance Keltrol®, Keltrol® T, Keltrol® Tf, Keltrol® Bt, Keltrol® Rd, Keltrol® Cg (Nutrasweet Kelco), Rhodicare® S and Rhodicare® H (Rhodia Chimie); starch derivatives, for instance Primogel® (Avebe); hydroxyethylcelluloses such as Cellosize® QP3L, QP4400H, QP30000H, HEC30000A and Polymer PCG10 (Amerchol), Natrosol® 250HHR, 250MR, 250M, 250HHXR, 250HHX, 250HR, HX (Hercules) and Tylose® H1000 (Hoechst); hydroxypropylcelluloses, for instance Klucel® EF, H, LHF, MF and G (Aqualon); carboxymethylcelluloses, for instance Blanose® 7M8/SF, refined 7M, 7LF, 7MF, 9M31F, 12M31XP, 12M31P, 9M31XF, 7H, 7M31, 7H3SXF (Aqualon), Aquasorb® A500 (Hercules), Ambergum® 1221 (Hercules), Cellogen® HP810A, HP6HS9 (Montello) and Primellose® (Avebe).

In other preferred embodiments, the at least one thickening agent are chosen from nonionic polymers which include, but are not limited to, nonionic cellulose derivatives such as hydroxyethylcelluloses modified by groups comprising at least one hydrophobic chain, such as alkyl, arylalkyl or alkylaryl groups, or their blends, and in which the alkyl groups are, for example, C8-C22 alkyl groups, such as the product Natrosol® Plus Grade 330 CS (C16 alkyls) sold by Aqualon or the product Bermocoll® EHM 100 sold by Berol Nobel.

Cellulose derivatives modified by alkylphenyl polyalkylene glycol ether groups may also be chosen, such as the product Amercell® Polymer HM-1500 sold by Amerchol.

In certain exemplary embodiments, the at least one thickening agent is chosen from cellulose derivatives, polysaccharides, gums, clays, fumed silica, acrylates, polyacrylamides, crosslinked polyacrylic acids, crosslinked acrylamide polymers and copolymers, crosslinked methacryloyloxyethltrimethyl-ammonium chloride homopolymers, and associative polymers. Said rheology-modifying agents may include, in particular embodiments, xanthan gum, gum arabic, ghatti gum, karaya gum, gum tragacanth, carrageenan gum, agar gum, carob gum, pectins, alginates, starches, hydroxy(C1-C6)alkylcelluloses, carboxy(C1-C6)alkylcelluloses, and mixtures thereof.

In preferred embodiments, the at least one thickening agent is nonionic or is an uncharged compound. Without intending to be bound by theory, it is believed that a thickening agent comprising a charged compound could adversely interact with the dyes compounds, thereby affecting the solubility and stability of the dye compounds in the compositions of the present disclosure.

In particularly preferred embodiments, the at least one thickening agent is chosen from xanthan gum, carrageenan gum, guar gum, and mixtures thereof.

The at least one thickening agent of the present disclosure may be employed in the composition of the present invention in an amount ranging from, for example, about 0.05 to about 10% by weight, preferably from about 0.1 to about 8% by weight and more preferably from about 0.25 to about 5% by weight, based on the total weight of the composition, including all ranges and subranges therebetween.

### Cosmetically Acceptable Medium

Besides the compounds indicated previously, a composition according to the invention comprises a cosmetically acceptable medium. In preferred embodiments, the cosmetically acceptable medium comprises water

The term “cosmetically acceptable medium” is intended to denote a medium that is particularly suitable for applying a composition according to the invention to the skin.

The cosmetically acceptable medium is generally adapted to the nature of the support onto which the composition is to be applied, and also to the form in which the composition is to be packaged.

A composition of the invention may be a dispersion or an emulsion.

A dispersion may be made as an aqueous phase or as an oily phase.

An emulsion may have an oily or aqueous continuous phase. Such an emulsion may be, for example, an inverse (W/O) emulsion or a direct (O/W) emulsion, or alternatively a multiple emulsion (W/O/W or O/W/O).

In the case of emulsions, direct (O/W) emulsions are preferred.

### Aqueous Phase

The composition according to the invention may comprise an aqueous phase.

The aqueous phase comprises water. A water that is suitable for use in the invention may be a floral water such as cornflower water and/or a mineral water such as Vittel water, Lucas water or La Roche Posay water and/or a spring water.

The aqueous phase may also comprise water-miscible organic solvents (at room temperature: 25° C.), for instance monoalcohols containing from 2 to 6 carbon atoms, such as ethanol or isopropanol; polyols especially containing from 2 to 20 carbon atoms, preferably containing from 2 to 10 carbon atoms and preferentially containing from 2 to 6 carbon atoms, such as glycerol, propylene glycol, butylene glycol, pentylene glycol, hexylene glycol, dipropylene glycol or diethylene glycol; glycol ethers (especially containing from 3 to 16 carbon atoms) such as mono-, di- or tripropylene glycol (C1-C4)alkyl ethers, mono-, di- or triethylene glycol (C1-C4)alkyl ethers, and mixtures thereof.

The aqueous phase may also comprise stabilizers, for example sodium chloride, magnesium dichloride or magnesium sulfate.

The aqueous phase may also comprise any water-soluble or water-dispersible compound that is compatible with an aqueous phase, such as gelling agents, film-forming polymers, thickeners or surfactants, and mixtures thereof.

In particular, a composition of the invention may comprise an aqueous phase in a content ranging from 1% to 80% by weight, especially from 5% to 50% and more particularly from 10% to 45% by weight, based on the total weight of the composition, including all ranges and subranges therebetween.

### Fatty Phase

A cosmetic composition in accordance with the present invention may comprise at least one liquid and/or solid fatty phase.

According to one embodiment, the composition according to the present invention is in the form of an emulsion.

In particular, a composition of the invention may comprise at least one liquid fatty phase, especially at least one oil as mentioned below.

The term “oil” means any fatty substance that is in liquid form at room temperature (20-25° C.) and at atmospheric pressure.

A composition of the invention may comprise a liquid fatty phase in a content ranging from about 1% to about 90%, in particular from about 5% to about 80%, in particular from about 10% to about 70% and more particularly from about 20% to about 50% by weight relative to the total weight of the composition.

The oily phase that is suitable for preparing the cosmetic compositions according to the invention may comprise hydrocarbon-based oils, silicone oils, fluoro oils or non-fluoro oils, or mixtures thereof.

The oils may be volatile or non-volatile.

They may be of animal, plant, mineral or synthetic origin.

The term “non-volatile oil” means an oil that remains on the skin at room temperature and atmospheric pressure. More specifically, a non-volatile oil has an evaporation rate strictly less than 0.01 mg/cm2/min.

To measure this evaporation rate, 15 g of oil or of oil mixture to be tested are placed in a crystallizing dish 7 cm in diameter, which is placed on a balance in a large chamber of about 0.3 m3 that is temperature-regulated, at a temperature of 25° C., and hygrometry-regulated, at a relative humidity of 50%. The liquid is allowed to evaporate freely, without stirring it, while providing ventilation by means of a fan (Papst-Motoren, reference 8550 N, rotating at 2700 rpm) placed in a vertical position above the crystallizing dish containing said oil or said mixture, the blades being directed towards the crystallizing dish, 20 cm away from the bottom of the crystallizing dish. The mass of oil remaining in the crystallizing dish is measured at regular intervals. The evaporation rates are expressed in mg of oil evaporated per unit of area (cm2) and per unit of time (minutes).

The term “volatile oil” means any non-aqueous medium that is capable of evaporating on contact with the skin or the lips in less than one hour, at room temperature and atmospheric pressure. The volatile oil is a cosmetic volatile oil, which is liquid at room temperature. More specifically, a volatile oil has an evaporation rate of between 0.01 and 200 mg/cm2/min, limits included.

For the purposes of the present invention, the term “silicone oil” means an oil comprising at least one silicon atom, and especially at least one Si—O group.

The term “fluoro oil” means an oil comprising at least one fluorine atom.

The term “hydrocarbon-based oil” means an oil mainly containing hydrogen and carbon atoms.

The oils may optionally comprise oxygen, nitrogen, sulfur and/or phosphorus atoms, for example in the form of hydroxyl or acid radicals.

### Volatile Oils

The volatile oils may be chosen from hydrocarbon-based oils containing from 8 to 16 carbon atoms, and especially C8-C16 branched alkanes (also known as isoparaffins), for instance isododecane (also known as 2,2,4,4,6-pentamethylheptane), isodecane and isohexadecane, for instance the oils sold under the trade names Isopar® or Permethyl®.

Volatile oils that may also be used include volatile silicones, for instance volatile linear or cyclic silicone oils, especially those with a viscosity of less than or equal to 8 centistokes (cSt) (8×10-6 m2/s), and especially containing from 2 to 10 silicon atoms and in particular from 2 to 7 silicon atoms, these silicones optionally comprising alkyl or alkoxy groups containing from 1 to 10 carbon atoms. As volatile silicone oils that may be used in the invention, mention may be made especially of dimethicones with viscosities of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

According to one embodiment, a composition of the invention may comprise from about 1% to about 80% by weight, or even from about 5% to about 70% by weight, or even from about 10% to about 60% by weight and especially from about 15% to about 50% by weight of volatile oil relative to the total weight of the composition.

### Non-Volatile Oils

The non-volatile oils may be chosen especially from non-volatile hydrocarbon-based, fluoro and/or silicone oils.

Non-volatile hydrocarbon-based oils that may especially be mentioned include:
hydrocarbon-based oils of animal origin, such as perhydrosqualene,
hydrocarbon-based oils of plant origin, such as phytostearyl esters, such as phytostearyl oleate, phytostearyl isostearate and lauroyl/octyldodecyl/phytostearyl glutamate (Ajinomoto, Eldew PS203), triglycerides formed from fatty acid esters of glycerol, in particular in which the fatty acids may have chain lengths ranging from C₄ to C₃₆ and especially from C₁₈ to C₃₆, these oils possibly being linear or branched, and saturated or unsaturated; these oils may especially be heptanoic or octanoic triglycerides, shea oil, alfalfa oil, poppy oil, winter squash oil, millet oil, barley oil, quinoa oil, rye oil, candlenut oil, passionflower oil, shea butter, aloe vera oil, sweet almond oil, peach stone oil, groundnut oil, argan oil, avocado oil, baobab oil, borage oil, broccoli oil, calendula oil, camelina oil, canola oil, carrot oil, safflower oil, flax oil, rapeseed oil, cotton oil, coconut oil, marrow seed oil, wheatgerm oil, jojoba oil, lily oil, macadamia oil, corn oil, meadowfoam oil, St John's Wort oil, monoi oil, hazelnut oil, apricot kernel oil, walnut oil, olive oil, evening primrose oil, palm oil, blackcurrant pip oil, kiwi seed oil, grapeseed oil, pistachio oil, winter squash oil, pumpkin oil, musk rose oil, sesame oil, soybean oil, sunflower oil, castor oil and watermelon seed oil, and mixtures thereof, or alternatively caprylic/capric acid triglycerides, such as those sold by the company Stearineries Dubois or those sold under the names Miglyol® 810, 812 and 818 by the company Dynamit Nobel,
linear or branched hydrocarbons of mineral or synthetic origin, such as liquid paraffins and derivatives thereof, petroleum jelly, polydecenes, polybutenes, hydrogenated polyisobutene such as Parleam, and squalane;
synthetic ethers containing from 10 to 40 carbon atoms;
oils of high molar mass, in particular having a molar mass ranging from about 400 to about 10 000 g/mol, in particular from about 650 to about 10 000 g/mol, in particular from about 750 to about 7500 g/mol and more particularly ranging from about 1000 to about 5000 g/mol. As oils of high molar mass that may be used in the present invention, mention may especially be made of oils chosen from:
lipophilic polymers,
silicone oils,
oils of plant origin, and
mixtures thereof,
optionally partially hydrocarbon-based and/or silicone fluoro oils, for instance fluorosilicone oils, fluoropolyethers and fluorosilicones as described in document EP-A-847 752;
silicone oils, for instance linear or cyclic non-volatile polydimethylsiloxanes (PDMS); polydimethylsiloxanes comprising alkyl, alkoxy or phenyl groups, which are pendant or at the end of a silicone chain, these groups containing from 2 to 24 carbon atoms; phenyl silicones, for instance phenyl trimethicones, phenyl dimethicones, phenyl trimethylsiloxy diphenyl siloxanes, diphenyl dimethicones, diphenyl methyldiphenyl trisiloxanes and 2-phenylethyl trimethylsiloxy silicates, and
mixtures thereof.

According to one particular embodiment, the fatty phase of the composition according to the invention can contain only volatile compounds.

According to another particular embodiment, the fatty phase of the composition according to the invention can contain non-volatile oils, preferably, in a total amount of not more than about 20% by weight, preferably, not more than about 10% by weight, or more preferably, not more than about 5% by weight, based on the total weight of the composition.

According to certain embodiments, the fatty substance of the present invention chosen from fatty alcohols and fatty acids are present in the fatty phase of the composition.

### Additives

A composition according to the invention may also comprise any additive usually used in the field under consideration, chosen, for example, from waxes, dispersants, antioxidants, essential oils, preserving agents, fragrances, neutralizers, antiseptics, UV-screening agents, cosmetic active agents, such as vitamins, moisturizers, emollients or collagen-protecting agents, and mixtures thereof.

It is a matter of routine operations for a person skilled in the art to adjust the nature and amount of the additives present in the compositions in accordance with the invention such that the desired cosmetic properties and stability properties thereof are not thereby affected.

A cosmetic composition of the invention may be in the form of a skin makeup product, in particular a foundation, a hot-cast foundation product, a body makeup product, a concealer, or an eyeshadow. It may be in the form of cream, lotion or soft paste.

A care composition according to the invention may in particular be a sunscreen or a moisturizing composition.

Preferably, the composition according to the invention is in the form of a fluid primer or base or a fluid foundation. When the composition is a foundation, it further contains at least one colorant as described above in an amount sufficient to provide additional color to or change the color of the skin.

In a particular embodiment, the composition is an emulsion. Such emulsions will generally be comprised of the liquid fatty phase (also known as an oil phase) and the aqueous phase as described earlier.

The table below comprises of materials that could be used in the composition, some materials already being described above and some introduced below:

| Product | |
|---|---|
| Form | Ingredient list |
| Oil-based | Isododecane, dimethicone, dimethiconol, diethyl sebacate, isodecyl pivalate, |
| | tris(ethylhexanoate) glyceryl, coconut oil, one or more photochromic compounds, |
| | fragrance, phenoxyethanol, tocopherol, jojoba seed oil |
| Water | TWEEN80, coconut oil, water, one or more photochromic compounds, fragrance |
| based | |
| Mousse | Cyclohexasiloxane, dimethicone, dimethicone/vinyl dimethicone crosspolymer, |
| | silica, acrylates copolymer, disodium stearoyl glutamate, aluminum hydroxide, |
| | coconut oil, one or more photochromic compounds, fragrance |
| Solid | Talc, dimethicone, HDI/trimethylol hexyllactone crosspolymer, silica, |
| powder | dimethicone/vinyl dimethicone crosspolymer, mica, titanium dioxide (CI 77891), |
| | one or more photochromic compounds, triethoxycaprylylsilane, ethylhexylglycerin, |
| | glyceryl caprylate, dimethiconol, iron oxides (CI 77492), aluminum hydroxide, |
| | alcohol, butylene glycol, iron oxides (CI 77491), methicone, tocopherol |
| Soft gel | Water, iota carrageenan, TWEEN80, coconut oil, one or more photochromic |
| | compounds |
| Serum | Glycerin, water, dimethicone, butylene glycol, myristyl alcohol, propylene glycol, |
| | cyclopentasiloxane, isopropyl myristate, cetyl ethylhexanoate, hydroxypropyl starch |
| | phosphate, perfume, steartrimonium chloride, guar hydroxypropyltrimonium |
| | chloride, sodium benzoate, cetrimonium chloride, laureth-23, laureth-3, |
| | polyquaternium-7, betaine, milk protein extract, panthenol, hydrolyzed collagen, |
| | Spirulina platensis extract, Corchorus olitorius leaf extract, disodium EDTA, |
| | tocopheryl acetate, C12-15 pareth-3, cocamidopropyl betaine, hydrolyzed keratin, |
| | hydrolyzed collagen PG-propyl methylsilanediol, hydrolyzed wheat protein, |
| | hydrolyzed corn protein, hydrolyzed soy protein, 1,2-hexanediol, glycine, glutamic |
| | acid, lysine, leucine, methionine, valine, serine, cysteine, aspartic acid, alanine, |
| | arginine, isoleucine, tyrosine, threonine, phenylalanine, proline, histidine, |
| | hydrolyzed silk, propanediol, Ceratonia siliqua (carob) fruit extract, Diospyros kaki |
| | fruit extract, Camellia japonica leaf extract, Myrothamnus flabellifolia leaf/stem |
| | extract, caprylyl glycol, citric acid, ascorbic acid, oat amino acids, wheat amino |
| | acids, soy amino acids, rice amino acids |
| Solid | Water, sodium laureth sulfate, sodium lauryl sulfate, cocamidopropyl betaine, |
| foam | sodium xylenesulfonate, glycol distearate, fragrance, sodium citrate, sodium |
| | chloride, dimethiconol, piroctone olamine, menthol, dimethicone, citric acid, sodium |
| | benzoate, guar hydroxypropyltrimonium chloride, tea-dodecylbenzenesulfonate, |
| | tetrasodium edta, sodium hydroxide, trideceth-10, magnesium nitrate, |
| | methylchloroisothiazolinone, triethylene glycol, propylene glycol, benzyl alcohol, |
| | magnesium chloride, methylisothiazolinone, copper sulfate, blue 1 |

The method may utilize any light based treatment device such as those found in, e.g. without limitation, U.S. Pat. No. 10,524,861 issued to Beerwerth et al. on Jan. 7, 2020.

### EXAMPLES/COMBINATIONS

A. A method for removing hair, the method comprising:
Applying a composition comprising a color changing compound to a treatment area and treating the treatment area with a light based treatment device.
B. The method of paragraph A, wherein the composition is in the form of a liquid, a solid, a semi-solid, a gel, or any other cosmetically acceptable form.
C. The method of any of paragraphs A or B, wherein the color changing compound comprises a photochromic compound, a light sensitive die, a thermochromic compound, or a combination thereof.
D. The method of any of paragraphs A to C, wherein the step of treating the treatment area with a light based treatment device further comprise treating the treatment area until the composition exhibits a Delta E of at least 0.5.
E. The method of paragraph D, wherein the composition exhibits a Delta E of at least 5.
F. The method of any of paragraphs A to E, wherein the color changing compound is a p-type photochromic dye.
G. The method of paragraph F, wherein the composition comprises a second photochromic dye.
H. The method of any of paragraphs A to G, wherein the light based treatment device emits an EMR having wavelengths in a range of 400 nm to 1400.
I. The method of any of paragraphs C to H, wherein the thermochromic compound is selected from the group comprising of leuco dyes, inorganic materials, or combinations thereof.
J. The method of any of paragraphs A to I, wherein the photochromic dye is de-activated during the step of treating the treatment area with a light based treatment device.
K. A kit comprising a light based treatment device and a composition comprising a color changing compound.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Throughout the specification, where the compositions, processes, kits, or apparatus are described as including components, steps, or materials, it is contemplated that the compositions, processes, or apparatus can also comprise, consist essentially of, or consist of, any combination of the recited components or materials, unless described otherwise. Component concentrations can be expressed in terms of weight concentrations, unless specifically indicated otherwise.

Combinations of components are contemplated to include homogeneous and/or heterogeneous mixtures, as would be understood by a person of ordinary skill in the art in view of the foregoing disclosure.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to anything disclosed or claimed herein or that it alone, or in any combination with any other reference or references, teaches, suggests or discloses anything disclosed or claimed herein. Further, to the extent that any meaning or definition of a term in this document conflicts with any meaning or definition of the same term in a document incorporated by reference, the meaning or definition assigned to that term in this document shall govern.

While particular forms of the present disclosure have been illustrated and described, it would be obvious to those skilled in the art that various other changes and modifications can be made without departing from the spirit and scope of the present disclosure. It is therefore intended to cover in the appended claims all such changes and modifications that are within the scope of the present disclosure.

1. A method for treating skin, the method comprising:
Applying a composition to a treatment area and
treating the treatment area with a light based treatment device;
wherein the composition comprises a color changing compound.

2. The method of claim 1, wherein the step of further comprises wiping the treatment area.

3. The method of claim 1, wherein the step of treating the treatment area with a light based treatment device further comprise treating the treatment area until the composition exhibits a Delta E of at least 0.5.

4. The method of claim 1, wherein the color changing compound comprises a photochromic compound, a light sensitive dye, a thermochromic compound, or a combination thereof.

5. The method of claim 4, wherein the thermochromic compound is selected from the group comprising of leuco dyes, inorganic materials, or combinations thereof.

6. The method of claim 4, wherein the photochromic compound is a p-type photochromic dye.

7. The method of claim 4, wherein the composition comprises a second photochromic dye.

8. The method of claim 1, wherein the light based treatment device emits an EMR having wavelengths in a range of 400 nm to 1400.

9. The method of claim 4, wherein the photochromic dye is activated during the step of treating the treatment area with a light based treatment device.

10. A method for removing hair, the method comprising:
Applying a composition comprising a color changing compound to a treatment area and treating the treatment area with a light based treatment device.

11. The method of claim 10, wherein the composition is in the form of a liquid, a solid, a semi-solid, a gel, or any other cosmetically acceptable form.

12. The method of claim 10, wherein the color changing compound comprises a photochromic compound, a light sensitive die, a thermochromic compound, or a combination thereof.

13. The method of claim 10, wherein the step of treating the treatment area with a light based treatment device further comprise treating the treatment area until the composition exhibits a Delta E of at least 0.5.

14. The method of claim 13, wherein the composition exhibits a Delta E of at least 5.

15. The method of claim 10, wherein the color changing compound is a p-type photochromic dye.

16. The method of claim 15, wherein the composition comprises a second photochromic dye.

17. The method of claim 10, wherein the light based treatment device emits an EMR having wavelengths in a range of 400 nm to 1400.

18. The method of claim 12, wherein the thermochromic compound is selected from the group comprising of leuco dyes, inorganic materials, or combinations thereof.

19. The method of claim 10, wherein the photochromic dye is de-activated during the step of treating the treatment area with a light based treatment device.

20. A kit comprising a light based treatment device and a composition comprising a color changing compound.
